(19) United States
(12) Patent Application Publication
(10) Pub. No.: US 2025/0381029 A1
(43) Pub. Date: Dec. 18, 2025

(54) PROSTHETIC VALVES WITH APEX COVERINGS

(71) Applicant: EDWARDS LIFESCIENCES CORPORATION, Irvine, CA (US)

(72) Inventor: Michael C. Murad, Lake Mathews, CA (US)

(73) Assignee: EDWARDS LIFESCIENCES CORPORATION, Irvine, CA (US)

(21) Appl. No.: 19/304,830

(22) Filed: Aug. 20, 2025

Related U.S. Application Data
(63) Continuation of application No. PCT/US2024/018205, filed on Mar. 1, 2024.
(60) Provisional application No. 63/450,713, filed on Mar. 8, 2023.

(51) Int. Cl. A61F 2/24; A61F 2/00; A61F 2/90

(52) U.S. Cl. CPC A61F 2/2418; A61F 2/2415; A61F 2002/0081; A61F 2/90; A61F 2220/0075; A61F 2250/006

(57) ABSTRACT

The present disclosure relates to a prosthetic valves that include an apex coverings attached to apices of their frames, configured to form atraumatic contact surfaces around the apices. In an example, an apex covering includes an opening through which the apex can be inserted into an inner pocket of the apex covering. The apex covering can define an outer surface that can be curved and relatively smooth. The apex covering can include a pair of flexible arms that can rest over angled struts extending from the apex, so as to increase contact area between the apex and the apex covering. Sutures can be optionally used to couples the apex covering to the frame, forming loops around the frame and grooves formed along the outer surface of the apex covering.

### CROSS-REFERENCE TO RELATED APPLICATIONS

This application is a continuation of International Application No. PCT/US2024/018205, filed Mar. 1, 2024, which claims the benefit of U.S. Provisional Application No. 63/450,713, filed Mar. 8, 2023, which is incorporated by reference herein.

### FIELD

The present disclosure relates to prosthetic valves that include apex coverings disposed over apices of the frame, the apex coverings configured to form an external atraumatic surface around the apices.

### BACKGROUND

Native heart valves, such as the aortic, pulmonary and mitral valves, function to assure adequate directional flow from, and to, the heart, and between the heart's chambers, to supply blood to the whole cardiovascular system. Various valvular diseases can render the valves ineffective and require replacement with artificial valves. Surgical procedures can be performed to repair or replace a heart valve. Conventional surgically implantable prosthetic valve typically include a leaflet assembly mounted within a relatively rigid support frame or ring. Components of the prosthetic valve are usually assembled with one or more biocompatible fabrics, and a fabric-covered sewing ring is provided around the valve for suturing to the tissue of the native leaflet.

Since surgeries are prone to an abundance of clinical complications, alternative less invasive techniques of delivering a prosthetic heart valve over a catheter and implanting it over the native malfunctioning valve have been developed over the years. Different types of prosthetic heart valves are known to date, including balloon expandable valve, self-expandable valves and mechanically-expandable valves. Different methods of delivery and implantation are also known, and may vary according to the site of implantation and the type of prosthetic valve. One exemplary technique includes utilization of a delivery apparatus for delivering a prosthetic valve in a crimped state, from an incision which can be located at the patient's femoral or iliac artery, toward the native malfunctioning valve. Once the prosthetic valve is properly positioned at the desired site of implantation, it can be expanded against the surrounding anatomy, such as an annulus of a native valve, and the delivery apparatus can be retrieved thereafter.

### SUMMARY

In some cases, prosthetic valves have frames with angled struts that form relatively pointy exposed apices. When such prosthetic valves are crimped over and around an inflatable balloon of a delivery apparatus, the exposed apices can occasionally damage, penetrate and tear the balloon wall material. Another issue with exposed apices includes the leading or inflow apices interacting with inner walls of a delivery shaft when axially advanced therethrough, or interacting with the balloon wall in configurations in which the valve is crimped over a portion of a balloon catheter proximal to the balloon, and is pushed toward and over the balloon upon reaching the site of implantation. Such interactions can scrape against or penetrate the delivery shaft, thereby causing damage to the shaft and potentially the vasculature, and/or cause degradation to the balloon which may result in inadequate inflation at the implantation site.

In one representative example, there is provided a prosthetic valve comprising a frame configured to transition between a crimped configuration and an expanded configuration. The frame comprises a plurality of intersecting struts and a plurality of apices. The prosthetic valve further comprises a plurality of apex coverings. Each of the apex coverings are attached to and covers a corresponding one of the plurality of apices. Each apex covering comprises: a main body extending between a first end and a second end, an opening formed at the second end, an inner pocket extending from the opening and sized to accommodate the corresponding apex therein, and a covering external surface facing away from the apex covered by apex covering. The opening is sized to allow insertion of the corresponding apex into the inner pocket.

In another representative example, there is provided a method of attaching an apex covering to an apex of a prosthetic valve. The method comprises providing an apex covering that comprises a main body extending between a first end and a second end, the second end comprising an opening from which an inner pocket or the main body extends toward a pocket closed end. The method further comprises inserting an apex of a frame of a prosthetic valve, through the opening, into the inner pocket.

In another representative example, there is provided a prosthetic valve. The prosthetic valve comprises a frame configured to transition between a crimped configuration and an expanded configuration. The frame comprises a plurality of intersecting struts and a plurality of apices. The prosthetic valve further comprises a plurality of apex coverings, each of the apex coverings attached to and covering a corresponding one of the plurality of apices. Each apex covering comprises: a closed first end; and a covering external surface facing away from the apex disposed inside the apex covering. Each apex can further include, in some examples, a closed second end opposite to the closed first end. The at least one apex covering can be optionally formed, in some examples, from a biodegradable material.

The aspects of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the invention will become more apparent from the following detailed description, which proceeds with reference to the accompanying figures.

### BRIEF DESCRIPTION OF THE FIGURES

Some examples of the invention are described herein with reference to the accompanying figures. The description, together with the figures, makes apparent to a person having ordinary skill in the art how some examples may be practiced. The figures are for the purpose of illustrative description and no attempt is made to show structural details of an example in more detail than is necessary for a fundamental understanding of the invention. For the sake of clarity, some objects depicted in the figures are not to scale.

In the Figures:

FIG. 1A is a perspective view of an exemplary prosthetic valve.

FIG. 1B is a perspective view of a frame of the prosthetic valve of FIG. 1A, with an enlarged region of the frame in the vicinity of an apex.

FIG. 2 shows an exemplary apex having a flat apex end surface.

FIG. 3 shows an exemplary M-shaped or W-shaped apex.

FIG. 4 shows an exemplary delivery apparatus carrying a prosthetic valve.

FIG. 5 shows a prosthetic valve crimped inside a crimping device.

FIG. 6 shows the prosthetic valve radially compressed onto and around a portion of an inflatable balloon.

FIG. 7 shows an exemplary frame of a prosthetic valve with the inflow and outflow apices curved radially inwards.

FIG. 8A shows an exemplary apex covering positioned in the vicinity of an apex, prior to attachment thereto.

FIG. 8B shows the apex covering of FIG. 8A disposed over the apex.

FIG. 9 shows an exemplary apex covering comprising grooves formed along the outer surface of the main body.

FIG. 10 shows the apex covering of FIG. 9 attached to the apex by suture loops extending around the grooves of the main body.

FIG. 11A shows an exemplary apex covering comprising a pair of arms extending from the main body, positioned in the vicinity of an apex, prior to attachment thereto.

FIG. 11B shows the apex covering of FIG. 11A disposed over the frame and its apex.

FIG. 12 shows an exemplary apex covering comprising grooves formed along the outer surface of the arms and the joints.

FIG. 13 shows the apex covering of FIG. 12 attached to the frame by a series of separate suture loops extending around the grooves of the arms and the joints.

FIG. 14 shows the apex covering of FIG. 12 attached to the frame by a series of loops formed by a single suture, the loops extending around the grooves of the arms and the joints.

FIG. 15A shows an exemplary apex covering positioned in the vicinity of an apex of the type shown in FIG. 3, prior to attachment thereto.

FIG. 15B shows the apex covering of FIG. 15A disposed over the apex.

FIG. 16 shows an apex covering formed around an apex, the apex covering axially extending between two closed ends thereof.

### DETAILED DESCRIPTION

For purposes of this description, certain aspects, advantages, and novel features of the examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present, or problems be solved. The technologies from any example can be combined with the technologies described in any one or more of the other examples. In view of the many possible examples to which the principles of the disclosed technology may be applied, it should be recognized that the illustrated examples are only preferred examples and should not be taken as limiting the scope of the disclosed technology.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the terms “have” or “includes” means “comprises”. Further, the terms “coupled”, “connected”, and “attached”, as used herein, are interchangeable and generally mean physically, mechanically, chemically, magnetically, and/or electrically coupled or linked and does not exclude the presence of intermediate elements between the coupled or associated items absent specific contrary language. As used herein, “and/or” means “and” or “or”, as well as “and” and “or”.

Directions and other relative references may be used to facilitate discussion of the drawings and principles herein, but are not intended to be limiting. For example, certain terms may be used such as “inner,” “outer,” “upper,” “lower,” “inside,” “outside,”, “top,” “bottom,” “interior,” “exterior,” “left,” right,” and the like. Such terms are used, where applicable, to provide some clarity of description when dealing with relative relationships, particularly with respect to the illustrated examples. Such terms are not, however, intended to imply absolute relationships, positions, and/or orientations. For example, with respect to an object, an “upper” part can become a “lower” part simply by turning the object over. Nevertheless, it is still the same part and the object remains the same.

The term “plurality” or “plural” when used together with an element means two or more of the element. Directions and other relative references (e.g., inner and outer, upper and lower, above and below, left and right, and proximal and distal) may be used to facilitate discussion of the drawings and principles herein but are not intended to be limiting.

The terms “axial direction,” “radial direction,” and “circumferential direction” have been used herein to describe the arrangement and assembly of components relative to the geometry of the frame of the prosthetic valve, or the geometry of an inflatable balloon that can be used to expand a prosthetic valve. Such terms have been used for convenient description, but the disclosed examples are not strictly limited to the description. In particular, where a component or action is described relative to a particular direction, directions parallel to the specified direction as well as minor deviations therefrom are included. Thus, a description of a component extending along an axial direction of the frame does not require the component to be aligned with a center of the frame; rather, the component can extend substantially along a direction parallel to a central axis of the frame.

As used herein, the terms “integrally formed” and “unitary construction” refer to a construction that does not include any welds, fasteners, or other means for securing separately formed pieces of material to each other.

As used herein, operations that occur “simultaneously” or “concurrently” occur generally at the same time as one another, although delays in the occurrence of operation relative to the other due to, for example, spacing between components, are expressly within the scope of the above terms, absent specific contrary language.

As used herein, terms such as “first,” “second,” and the like are intended to serve as respective labels of distinct components, steps, etc. and are not intended to connote or imply a specific sequence or priority. For example, unless otherwise stated, a step of performing a second action and/or of forming a second component may be performed prior to a step of performing a first action and/or of forming a first component.

As used herein, the term “substantially” means the listed value and/or property and any value and/or property that is at least 75% of the listed value and/or property. Equivalently, the term “substantially” means the listed value and/or property and any value and/or property that differs from the listed value and/or property by at most 25%. For example, “at least substantially parallel” refers to directions that are fully parallel, and to directions that diverge by up to 22.5 degrees.

In the present disclosure, a reference numeral that includes an alphabetic label (for example, “a,” “b,” “c,” etc.) is to be understood as labeling a particular example of the structure or component corresponding to the reference numeral. Accordingly, it is to be understood that components sharing like names and/or like reference numerals (for example, with different alphabetic labels or without alphabetic labels) may share any properties and/or characteristics as disclosed herein even when certain such components are not specifically described and/or addressed herein.

Throughout the figures of the drawings, different superscripts for the same reference numerals are used to denote different examples of the same elements. Examples of the disclosed devices and systems may include any combination of different examples of the same elements. Specifically, any reference to an element without a superscript may refer to any alternative example of the same element denoted with a superscript. In order to avoid undue clutter from having too many reference numbers and lead lines on a particular drawing, some components will be introduced via one or more drawings and not explicitly identified in every subsequent drawing that contains that component.

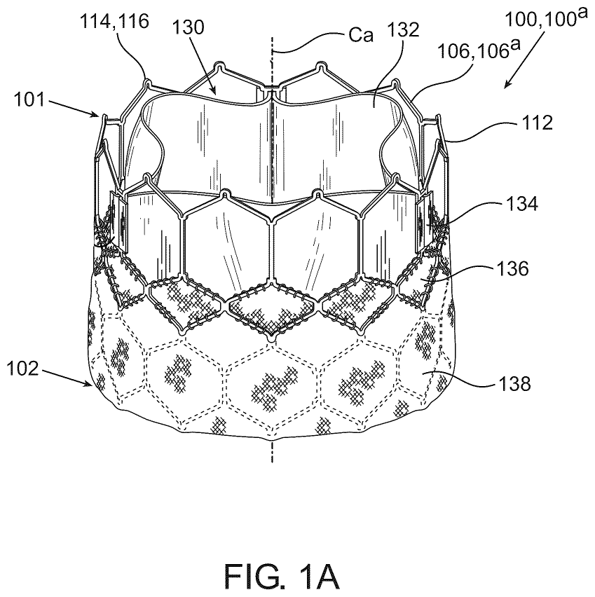

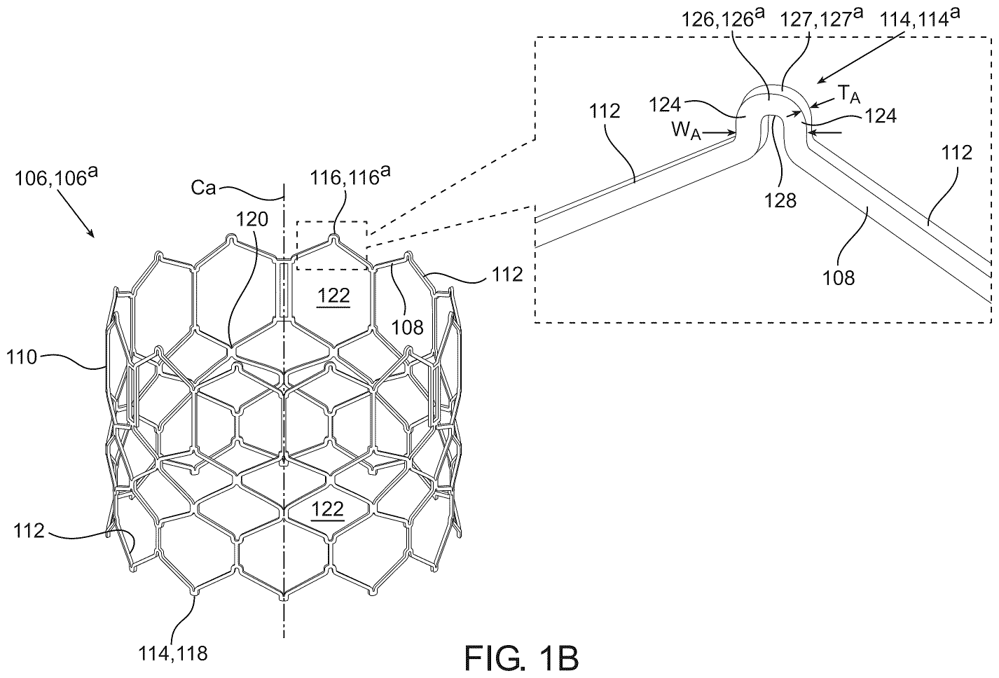

FIG. 1A shows a perspective view of one example of a prosthetic valve 100. FIG. 1B shows a frame 106 of the prosthetic valve 100 of FIG. 1A without any other soft components attached thereto. The term “prosthetic valve”, as used herein, refers to any type of a prosthetic valve deliverable to a patient's target site over a catheter, which is radially expandable and compressible between a radially compressed, or crimped, state, and a radially expanded state. Thus, the prosthetic valves can be crimped on or retained by an implant delivery apparatus (not shown) in the radially compressed state during delivery, and then expanded to the radially expanded state once the prosthetic valve reaches the implantation site. The expanded state may include a range of diameters to which the valve may expand, between the compressed state and a maximal diameter reached at a fully expanded state. Thus, a plurality of partially expanded states may relate to any expansion diameter between radially compressed or crimped state, and maximally expanded state.

Any of the prosthetic valves disclosed herein are adapted to be implanted in the native aortic annulus, although in other embodiments they can be adapted to be implanted in the other native annuluses of the heart (e.g., the pulmonary, mitral, and tricuspid valves). The disclosed prosthetic valves also can be implanted within vessels communicating with the heart, including a pulmonary artery (for replacing the function of a diseased pulmonary valve, or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For instance, in some examples, the disclosed prosthetic valves can be implanted within a docking device implanted within the pulmonary artery for replacing the function of a diseased pulmonary valve, such as disclosed in U.S. Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Publication No. 2019/0000615, which is incorporated herein by reference.

It is understood that the prosthetic valves disclosed herein may be used with a variety of implant delivery apparatuses. Balloon expandable valves generally involve a procedure of inflating a balloon within a prosthetic valve, thereby expanding the prosthetic valve within the desired implantation site. Once the valve is sufficiently expanded, the balloon is deflated and retrieved along with a delivery apparatus (not shown). Self-expandable valves include a frame that is shape-set to automatically expand as soon an outer retaining shaft or capsule (not shown) is withdrawn proximally relative to the prosthetic valve. Mechanically expandable valves are a category of prosthetic valves that rely on a mechanical actuation mechanism for expansion. The mechanical actuation mechanism usually includes a plurality of expansion and locking assemblies (such as the prosthetic valves described in U.S. Pat. No. 10,603,165, International Application No. PCT/US2021/052745 and U.S. Provisional Application Nos. 63/085,947 and 63/209,904, each of which is incorporated herein by reference in its entirety), releasably coupled to respective actuation assemblies of a delivery apparatus, controlled via a handle (not shown) for actuating the expansion and locking assemblies to expand the prosthetic valve to a desired diameter. The expansion and locking assemblies may optionally lock the valve's diameter to prevent undesired recompression thereof, and disconnection of the actuation assemblies from the expansion and locking assemblies, to enable retrieval of the delivery apparatus once the prosthetic valve is properly positioned at the desired site of implantation.

FIGS. 1A-1B show an example of a prosthetic valve 100, which can be a balloon expandable valve, illustrated in an expanded state. The prosthetic valve 100 can comprise an outflow end 101 and an inflow end 102. In some instances, the outflow end 101 is the proximal end of the prosthetic valve 100, and the inflow end 102 is the distal end of the prosthetic valve 100. Alternatively, depending for example on the delivery approach of the valve, the outflow end can be the distal end of the prosthetic valve, and the inflow end can be the distal end of the proximal valve.

The term “proximal”, as used herein, generally refers to a position, direction, or portion of a device or a component of a device, which is closer to the user (for example, closer to an operator of a delivery apparatus utilized during an implantation procedure) and farther away from the implantation site.

The term “distal”, as used herein, generally refers to a position, direction, or portion of a device or a component of a device, which is farther away from the user and closer to the implantation site.

The term “outflow”, as used herein, refers to a region of the prosthetic valve through which the blood flows through and out of the prosthetic valve 100.

The term “inflow”, as used herein, refers to a region of the prosthetic valve through which the blood flows into the prosthetic valve 100.

In the context of the present application, the terms “lower” and “upper” are used interchangeably with the terms “inflow” and “outflow”, respectively. Thus, for example, the lower end of the prosthetic valve is its inflow end and the upper end of the prosthetic valve is its outflow end.

In the context of the present application, the terms “lower” and “upper” are used interchangeably with the terms “distal to” and “proximal to”, respectively. Thus, for example, a lowermost component can refer to a distal-most component, and an uppermost component can similarly refer to a proximal-most component.

The terms “longitudinal” and “axial”, as used herein, refer to an axis extending in the proximal and distal directions, unless otherwise expressly defined.

The prosthetic valve 100 comprises an annular frame 106 movable between a radially compressed configuration and a radially expanded configuration, and a valvular structure 130 mounted within the frame 106. The frame comprises an inner surface 108, defines as the surface facing a central axis Ca of the prosthetic valve, and an opposite outer surface 110 facing away from the central axis Ca. The frame 106 can be made of various suitable materials, including plastically-deformable materials such as, but not limited to, stainless steel, a nickel based alloy (e.g., a cobalt-chromium or a nickel-cobalt-chromium alloy such as MP35N alloy), polymers, or combinations thereof. When constructed of a plastically-deformable materials, the frame 106 can be crimped to a radially compressed state on a balloon catheter, and then expanded inside a patient by an inflatable balloon or equivalent expansion mechanism. Alternatively or additionally, the frame 106 can be made of shape-memory materials such as, but not limited to, nickel titanium alloy (e.g., Nitinol). When constructed of a shape-memory material, the frame 106 can be crimped to a radially compressed state and restrained in the compressed state by insertion into a shaft or equivalent mechanism of a delivery apparatus.

In the example illustrated in FIGS. 1A-1B, the frame 106 is an annular, stent-like structure comprising a plurality of interconnected struts 112. In this application, the term “strut” encompasses axial struts, angled struts, laterally extendable struts, commissure windows, commissure support struts, support posts, and any similar structures described by U.S. Pat. Nos. 7,993,394 and 9,393,110, which are incorporated herein by reference. A strut 112 may be any elongated member or portion of the frame 106. The frame 106 can include a plurality of strut rungs that can collectively define one or more rows of cells 122. The frame 106 can have a cylindrical or substantially cylindrical shape having a constant diameter from the inflow end 102 to the outflow end 101 as shown, or the frame can vary in diameter along the height of the frame, as disclosed in U.S. Pat. No. 9,155,619, which is incorporated herein by reference.

The end portions of the struts 112 are forming apices 114 at the inflow and outflow ends of the valve, including outflow apices 116 at the outflow end 101 and inflow apices 118 at the inflow end 102. Each apex 114 is formed at a junction between two angled struts 112 at either the inflow end 102 or the outflow end 101. FIGS. 1A-1B depict an exemplary frame design with apices 114 that form a U-shaped bend between the two angled struts 112. The struts 112 can intersect at additional junctions 120 formed between the outflow apices 116 and the inflow apices 118. The junctions 120 can be equally or unequally spaced apart from each other, and/or from the apices 116, 118, between the outflow end 101 and the inflow end 102.

The struts 112 can include a plurality of angled struts and vertical or axial struts. FIGS. 1A-1B show an exemplary prosthetic valve 100 that can be representative of, but is not limited to, a balloon expandable prosthetic valve. The frame 106 of the prosthetic valve 100 illustrated in FIG. 1B comprises rungs of angled struts and axial struts disposed between some of the rungs of the angled struts. In such implementations of the frame, the struts can be pivotable or bendable relative to each other, so as to permit frame expansion or compression. For example, the frame 106 can be formed from a single piece of material, such as a metal tube, via various processes such as, but not limited to, laser cutting, electroforming, and/or physical vapor deposition, while retaining the ability to collapse/expand radially in the absence of hinges and like.

A valvular structure 130 can include a plurality of leaflets 132 (e.g., three leaflets), positioned at least partially within the frame 106, and configured to regulate flow of blood through the prosthetic valve 100 from the inflow end 102 to the outflow end 101. While three leaflets 132 arranged to collapse in a tricuspid arrangement, are shown in the example illustrated in FIG. 1A, it will be clear that a prosthetic valve 100 can include any other number of leaflets 132. Adjacent leaflets 132 can be arranged together to form commissures 134 that are coupled (directly or indirectly) to respective portions of the frame 106, thereby securing at least a portion of the valvular structure 130 to the frame 106. The leaflets 132 can be made from, in whole or part, biological material (e.g., pericardium), bio-compatible synthetic materials, or other such materials. Further details regarding transcatheter prosthetic valves, including the manner in which the valvular structures 130 can be coupled to the frame 106 of the prosthetic valve 100, can be found, for example, in U.S. Pat. Nos. 6,730,118, 7,393,360, 7,510,575, 7,993,394, 8,652,202, and 11,135,056, all of which are incorporated herein by reference in their entireties.

In some examples, the prosthetic valve 100 can comprise at least one skirt or sealing member. FIG. 1A shows an example of a prosthetic valve 100 that includes an inner skirt 136, which can be secured to the inner surface 108 of the frame 106. Such an inner skirt 136 can be configured to function, for example, as a sealing member to prevent or decrease perivalvular leakage. An inner skirt 136 can further function as an anchoring region for valvular structure 130 to the frame 106, and/or function to protect the leaflets 132 against damage which may be caused by contact with the frame 106, for example during valve crimping or during working cycles of the prosthetic valve 100. The inner skirt 136 can be disposed around and attached to the inner surface 108 of frame 106, wherein the valvular structure 130 can be sutured to the inner skirt 136 along a scalloped line. The inner skirt 136 can be coupled to the frame 106 via sutures or another form of coupler.

The prosthetic valve 100 can comprise, in some examples, an outer skirt 138 mounted on the outer surface 110 of frame 106, configure to function, for example, as a sealing member retained between the frame 106 and the surrounding tissue of the native annulus against which the prosthetic valve is mounted, thereby reducing risk of paravalvular leakage (PVL) past the prosthetic valve 100. The outer skirt 138 can be coupled to the frame 106 via sutures or another form of coupler.

Any of the inner skirt 136 and/or outer skirt 138 can be made of various suitable biocompatible materials, such as, but not limited to, various synthetic materials (e.g., PET) or natural tissue (e.g. pericardial tissue). In some cases, the inner skirt 136 can be formed of a single sheet of material that extends continuously around the inner surface 108 of frame 106. In some cases, the outer skirt 138 can be formed of a single sheet of material that extends continuously around the outer surface 110 of frame 106.

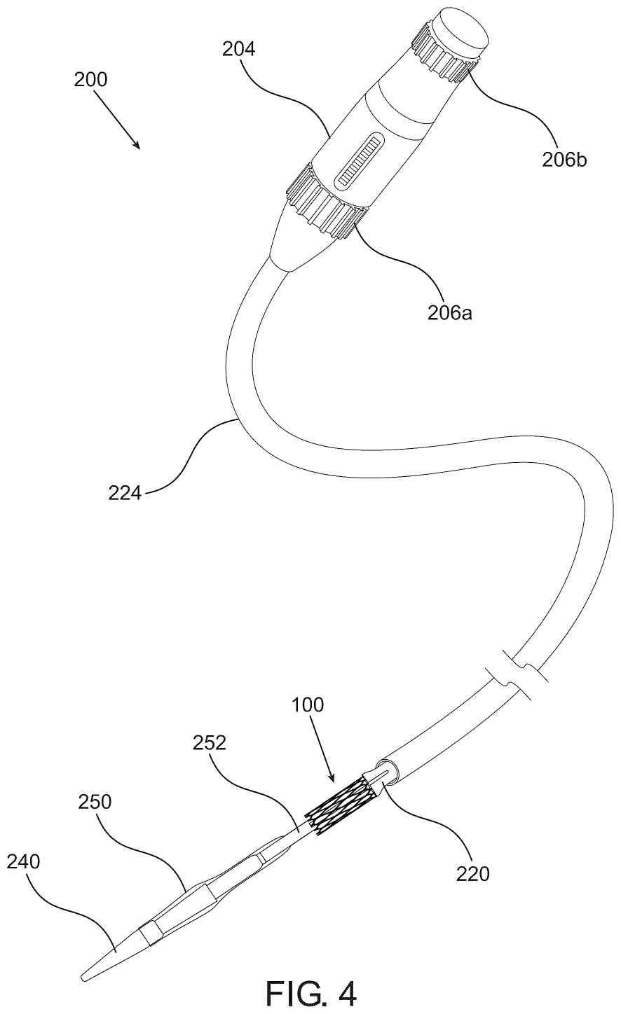

FIG. 4 illustrate a delivery apparatus 200, according to an exemplary configuration, adapted to deliver a balloon expandable prosthetic valve 100 described herein (e.g., prosthetic valve 100^{a} or 100^{b}). It should be understood that the delivery apparatus 200 can be used to implant prosthetic devices other than prosthetic valves, such as stents or grafts.

The delivery apparatus 200 includes a handle 204 and a balloon catheter 252 having an inflatable balloon 250 mounted on its distal end. The prosthetic valve 100 can be carried in a crimped state over the balloon catheter 252. Optionally, an outer delivery shaft 224 can concentrically extend over the balloon catheter 252, and a push shaft 220 can be disposed over the balloon catheter 252, optionally between the balloon catheter 252 and the outer delivery shaft 224.

The outer delivery shaft 224, the push shaft 220, and the balloon catheter 252, can be configured to be axially movable relative to each other. For example, a proximally oriented movement of the outer delivery shaft 224 relative to the balloon catheter 252, or a distally oriented movement of the balloon catheter 252 relative to the outer delivery shaft 224, can expose the prosthetic valve 100 from the outer delivery shaft 224. The delivery apparatus 200 can further include a nosecone 240 carried by a nosecone shaft 238 (hidden from view in FIG. 4, shown in FIG. 6) extending through a lumen of the balloon catheter 252.

The proximal ends of the balloon catheter 252, the outer delivery shaft 224, the push shaft 220, and optionally the nosecone shaft 238, can be coupled to the handle 204. During delivery of the prosthetic valve 100, the handle 204 can be maneuvered by an operator (e.g., a clinician or a surgeon) to axially advance or retract components of the delivery apparatus 200, such as the nosecone shaft 238, the balloon catheter 252, the outer delivery shaft 224, and/or the push shaft 220, through the patient's vasculature, as well as to inflate the balloon 250 mounted on the balloon catheter 252, so as to expand the prosthetic valve 100, and to deflate the balloon 250 and retract the delivery apparatus 200 once the prosthetic valve 100 is mounted in the implantation site.

The handle 204 can include a steering mechanism configured to adjust the curvature of the distal end portion of the delivery apparatus 200. In the illustrated example, the handle 204 includes an adjustment member, such as the illustrated rotatable knob 206a, which in turn is operatively coupled to the proximal end portion of a pull wire. The pull wire can extend distally from the handle 204 through the outer delivery shaft 224 and has a distal end portion affixed to the outer delivery shaft 224 at or near the distal end of the outer delivery shaft 224. Rotating the knob 206a can increase or decrease the tension in the pull wire, thereby adjusting the curvature of the distal end portion of the delivery apparatus 200. Further details on steering or flex mechanisms for the delivery apparatus can be found in U.S. Pat. No. 9,339,384, which is incorporated by reference herein. The handle 204 can further include an adjustment mechanism including an adjustment member, such as the illustrated rotatable knob 206b. The adjustment mechanism can be configured to adjust the axial position of the push shaft 220 relative to the balloon catheter.

The prosthetic valve 100 can be carried by the delivery apparatus 200 during delivery in a crimped state, and expanded by balloon inflation to secure it in a native heart valve annulus. In an exemplary implantation procedure, the prosthetic valve 100 is initially crimped over the balloon catheter 252, proximal to the inflatable balloon 250. Because prosthetic valve 100 is crimped at a location different from the location of balloon 250, prosthetic valve 100 can be crimped to a lower profile than would be possible if it was crimped on top of balloon 250. This lower profile permits the clinician to more easily navigate the delivery apparatus 200 (including crimped prosthetic valve 100) through a patient's vasculature to the treatment location. The lower profile of the crimped prosthetic valve is particularly helpful when navigating through portions of the patient's vasculature which are particularly narrow, such as the iliac artery.

The balloon 250 can be secured to balloon catheter 252 at its balloon proximal end, and to either the balloon catheter 252 or the nosecone 240 at its distal end. The distal end portion of the push shaft 220 is positioned proximal to the outflow end (e.g., outflow end 101) of the prosthetic valve 100.

When reaching the site of implantation, and prior to balloon inflation, the push shaft 220 can be advanced distally, allowing its distal end portion to contact and push against the outflow end of prosthetic valve 100, pushing the valve 100 distally therewith. The distal end of push shaft 220 is dimensioned to engage with the outflow end of the prosthetic valve 100 in a crimped configuration of the valve. In some implementations, the distal end portion of the push shaft 220 can be flared radially outward, to terminate at a wider-diameter that can contact the prosthetic valve 100 in its crimped state. Push shaft 220 can then be advanced distally, pushing the prosthetic valve 100 therewith, until the crimped prosthetic valve 100 is disposed around the balloon 250, at which point the balloon 250 can be inflated to radially expand the prosthetic valve 100. Once the prosthetic valve 100 is expanded to its functional diameter within a native annulus, the balloon 250 can be deflated, and the delivery apparatus 200 can be retrieved from the patient's body.

In particular implementations, the delivery apparatus 200 with the prosthetic valve 100 assembled thereon, can be packaged in a sterile package that can be supplied to end users for storage and eventual use. In particular implementations, the leaflets of the prosthetic valve (typically made from bovine pericardium tissue or other natural or synthetic tissues) are treated during the manufacturing process so that they are completely or substantially dehydrated and can be stored in a partially or fully crimped state without a hydrating fluid. In this manner, the package containing the prosthetic valve 100 and the delivery apparatus 200, can be free of any liquid. Methods for treating tissue leaflets for dry storage are disclosed in U.S. Pat. Nos. 8,007,992 and 8,357,387, both of which documents are incorporated herein by reference.

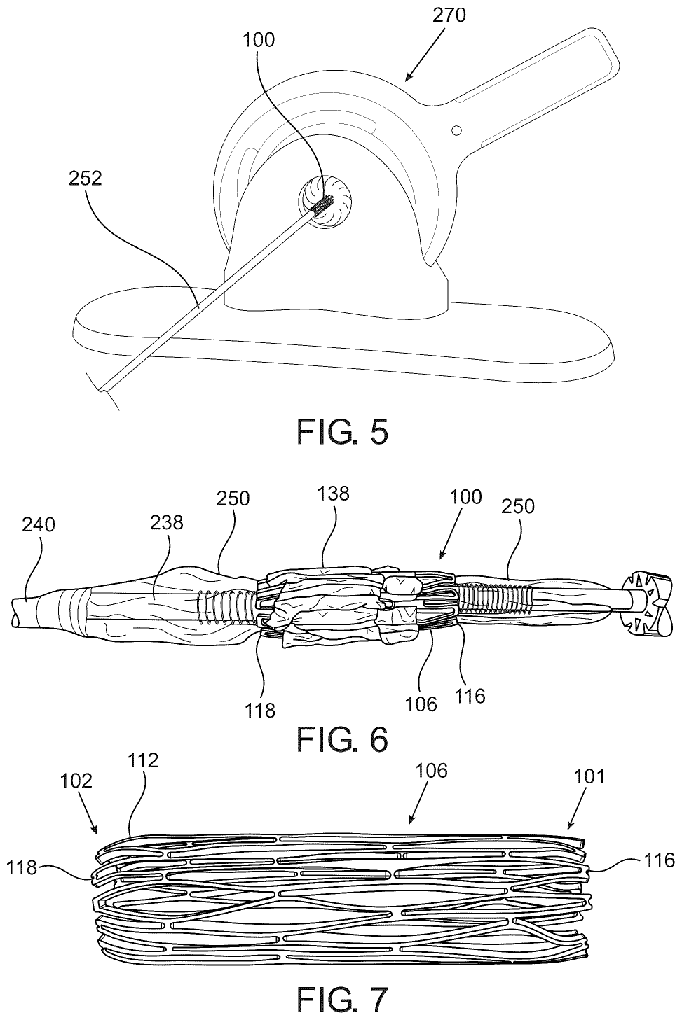

While described above and shown in FIG. 4 to be crimped proximal to the balloon 250, so as to be pushed by a push shaft 220 toward and over the balloon 250 prior to balloon inflation, in some implementation, the delivery apparatus 200 does not necessarily include a push shaft 220, and the prosthetic valve 100 can be crimped directly over the deflated balloon 250 prior to introduction into the patient's body. A prosthetic valve 100 may be assembled in a radially expanded state, as shown in FIGS. 1A-1B. Prior to insertion into the patient's body, a crimping device 270, as shown in FIG. 5, can be used to crimp the prosthetic valve 100 to the compressed configuration, which can be then stored in this configuration up to utilization thereof for implantation into the patient's body.

As shown in FIG. 5, the prosthetic valve 100 can be inserted into a receiving opening of a crimping device 270 while the valve is placed over a catheter, which, in the case of a balloon expandable prosthetic valve, can be a balloon catheter 252. The valve 100 can be crimped either proximal to the balloon 250, as shown in FIG. 4, or directly over the balloon 250, as shown in FIG. 6 and will be further described below. A crimping device 270 can be designed to translate a rotational movement of its housing to convergence of a plurality of plates thereof around the inner receiving opening into which the prosthetic valve 100 is inserted, thereby crimping the prosthetic valve 100 onto the balloon catheter 252. In a specific example, the crimping device 270 can include twelve plates configured to converge to crimp a prosthetic valve 100. Nevertheless, any other type of a crimping device 270 known in the art can be utilized for compressing the prosthetic valve 100.

Various exemplary implementations for various components and devices can be referred to, throughout the specification, with superscripts, for ease of explanation of features that refer to such exemplary implementations. It is to be understood, however, that any reference to structural or functional features of any assembly, apparatus or component, without a superscript, refer to these features being commonly shared by all specific exemplary implementations that can be also indicated by superscripts. In contrast, features emphasized with respect to an exemplary implementation of any assembly, apparatus or component, referred to with a superscript, may be optionally shared by some but not necessarily all other exemplary implementations. For example, prosthetic valve 100^{a} is an exemplary implementation of prosthetic valve 100, and thus includes all of the features described for prosthetic valve 100 throughout the current disclosure, except that while a prosthetic valve 100 can include any type or shape of apices 114, prosthetic valve 100^{a} includes generally U-shaped apices 114^{a}, such as outflow apices 116^{a} and/or inflow apices 118^{a}.

In some examples, as shown in FIG. 1B, each apex 114 can have two side portions 124 that curve or bend axially outward from the angled struts 112 to which it is connected and an end portion 126 that extends between the two side portions 124 of the apex 114, The end portion 126 defines an apex end surface 127, which can be disposed normal to the central longitudinal axis Ca in some examples, and an inner depression 128 opposite to the apex end surface 127. The side portions 124 can extend in a direction that is parallel to the central axis Ca. In some examples, as shown in FIG. 1A, the end portion 126^{a} of an apex 114^{a} can include a curved apex end surface 127^{a}, which can be slightly convex or even dome-shaped, and an inner depression 128 that can be arcuate or curved in shape. Each apex 114^{a} can be curved, such as by having two bends, at its end portion 126, and further include two bends at the side portions 124 (e.g., one at the junction between each side portion 124 and angled strut 112). In this way, the apices 114^{a} can be U-shaped.

As further shown in FIG. 1B, apex 114 defines an apex width W_{A} in a circumferential direction of the frame, defined as the distance between the farthest edges of side portion 124, and an apex thickness T_{A} measured at the apex 114 between the frame inner surface 108 and the frame outer surface 110.

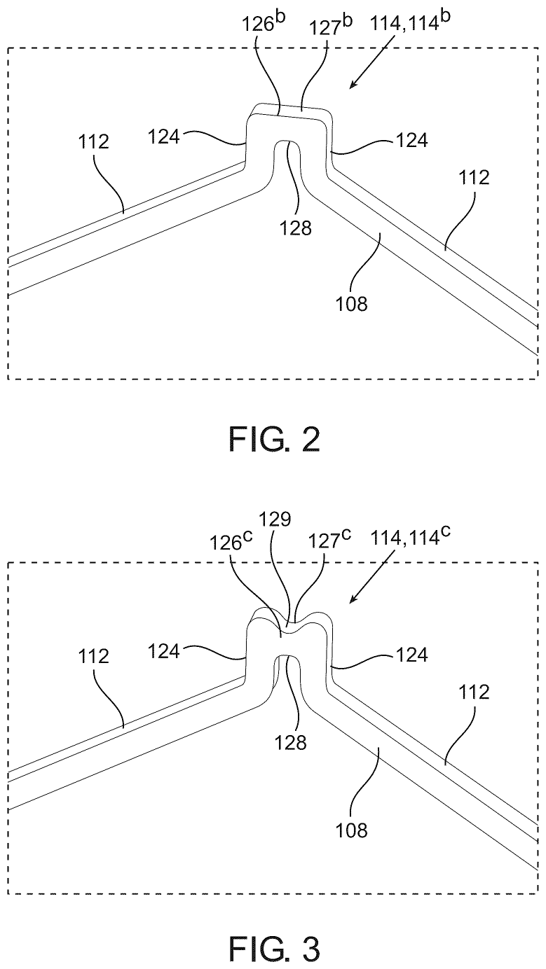

FIG. 2 shows an exemplary apex 114^{b} of a prosthetic valve 100^{b} (only a portion of the valve and a single apex is shown in FIG. 2). Prosthetic valve 100^{b} is an exemplary implementation of prosthetic valve 100, and thus includes all of the features described for prosthetic valve 100 throughout the current disclosure, except that the prosthetic valve 100^{b} includes apices 114^{b}, such as outflow apices 116^{b} and/or inflow apices 118^{b}. Apex 114^{b} can be similar in all respects to apex 114^{a}, except that while the apex 114^{a} includes a relatively curved apex end surface 127^{a}, the apex end surface 127^{b} of apex 114^{b} is relatively flat.

While U-shaped apices such as apices 114^{a} or apices 114^{b} can distribute stresses at the apices 114 across the angled struts 112 to which they are connected, the small dimensions of the apices causes their end portions 126 to be relatively sharp, relative to other flexible or soft components against which such apices 114 can be pressed or pushed, even when the end portions 126 are formed as relatively flat or rounded.

FIG. 3 shows an exemplary apex 114^{c} of a prosthetic valve 100^{c} (only a portion of the valve and a single apex is shown in FIG. 2). Prosthetic valve 100^{c} is an exemplary implementation of prosthetic valve 100, and thus includes all of the features described for prosthetic valve 100 throughout the current disclosure, except that the prosthetic valve 100 includes apices 114^{c}, such as outflow apices 116^{c} and/or inflow apices 118^{c}. Apex 114^{c} can be similar in all respects to any one of apex 114^{a} of 114^{b}, except that the apex end surface 127^{b} further comprises a notch 129 arranged between side portion 124^{c}. The notch 129 opens away from the inner depression 128, resulting in a W-shaped apex formation.

FIG. 6 shows the prosthetic valve 100 radially compressed onto and around a portion of inflatable balloon 250. In some examples, as shown in FIG. 6, the prosthetic valve 100 can be crimped onto balloon 250 with the inflow end 102 facing a nosecone 240. In other examples, the prosthetic valve 100 can be crimped onto balloon 250 with the outflow end 101 facing a nosecone 240.

In such a configuration, at least the apices 118 at the inflow end 102 are exposed and can damage, and in extreme cases, even puncture, the balloon materials as it is forcibly crimped over the balloon 250. The extent of damage to balloon material may depend on the size and shape of the apices 114, as well the profile of inflow end 102 and/or outflow end 101 of the frame 106 when crimped. Some exemplary frames can include pointed apices that can pose higher risk of puncturing the balloon when forcibly crimped thereover. However, even when provided as U-shaped apices, the small dimensions of such apices can still result in relatively sharp end portions that can damage the balloon 250. W-shaped apices, such as apices 114^{c}, can exhibit even sharper end portions defined by narrower ends of the side portions 124 on both sides of a notch 129.

The exposed apices 114 can also result in abrasion of the apices 114 against, or penetration of the apices 114 into, an inner wall of a delivery shaft (e.g., outer delivery shaft 224) when the prosthetic valve is advanced therethrough, or abrasion against an introduction sheath (not shown) through which the delivery apparatus 200 can travel en route to the implantation site (e.g., during delivery of the prosthetic valve 100 to the target implantation site). For example, during an implantation procedure when the prosthetic valve 100 is radially compressed (e.g., crimped) around the delivery apparatus 200, the prosthetic valve 100 can be pushed through an inner lumen of the delivery sheath and the apices 114 can contact, puncture, or tear the walls of the delivery sheath.

In some implementations, when the prosthetic valve 100 is crimped over the balloon catheter 252 proximal to the balloon 250, the exposed apices 114 can still interact with the balloon 250, such that upon reaching the implantation site, when the prosthetic valve 100 is pushed by the push shaft 220 over the balloon 250, the inflow apices 118 can interact with the balloon 250 (e.g., scrape against or resist movement of the valve over the balloon).

Moreover, in some implementations, the frame can be shaped so as to orient the apices 114 radially inwards (i.e., towards central axis Ca) during crimping, which can increase the risk of inflicting damage to the balloon 250. FIG. 7 shows an exemplary configuration of a frame in a compressed or state, which may occur after crimping the prosthetic valve, such as by crimping device 270, around the balloon. As shown, the frame in the exemplary configuration illustrated in FIG. 7 can be designed such that the inflow end 102 and the outflow end 101 are curved radially inward, while the remainder of the frame 106 can be arranged approximately parallel with the central axis Ca.

In some examples, as shown in FIG. 7, the plurality of both inflow apices 118 and outflow apices 116 are curved inward, toward the central axis Ca. However, in some example, the angle of inwards curvature of the outflow apices 116 may be less than that of the inflow apices 118. In some examples, only one end of the frame, such as the inflow end 102, is curved inwards.

While curving apices 114 radially inwards in a crimped state of the valve can mitigate the risk of the apices 114 abrading against or puncturing through inner walls of a delivery shaft during axial advancement within a lumen of the shaft, this configuration results in the apices 114 pointing toward the balloon 250 during crimping, which in turn can increase the risk of damaging the balloon 250 or even puncturing and tearing its wall material.

Another risk introduced by exposed apices of a prosthetic valve relates to inflow apices 118 that may contact an inner wall of another component of the delivery apparatus 200, such as an inner wall of a catheter or sheath in which the prosthetic valve 100 may reside during passage of the delivery apparatus 200 through the patient's vasculature. For example, an introducer sheath (not shown) can be arranged within a patient's blood vessel, extending along a portion of a length of the patient's blood vessel, in route to the target site of implantation. The introducer sheath can assume a curved profile throughout its length in the patient's body, including multiple curves or bends having different angles that are oriented at different directions, based on an architecture of the patient's vasculature. The delivery apparatus 200 can then be inserted into the introducer sheath and routed through it toward the target site of implantation.

When passing through curved region of the introducer sheath, the inflow end 101 of the prosthetic valve 100, including exposed inflow apices 118, can come into contact with the inner wall of the sheath. Direct contact between the inflow apices 118 and the sheath may cause degradation of the sheath. Furthermore, if the inflow apices 118 are pressed against the inner wall of the sheath with a great enough force at specific angles, the apices 118 may puncture the inner wall of the sheath. In some cases, contact of the relatively stiff and sharp apices 118 with the softer (or less stiff) introducer sheath may result in increased friction between the inflow apices 118 and the inner wall of the sheath, thereby resulting in adherence between the apices and the sheath (making it more difficult to advance the valve through the sheath the target implantation site) and potential damage to the sheath. Additionally, due to a relatively small contact area of the apices 118 (e.g., creating relatively sharp apices) against the sheath, the sheath may become degraded or damaged during contact with the inflow apices 118.

While described with respect to an introducer sheath, it is to be understood that exposed inflow apices 118 contacting an inner wall of any other shaft or catheter surrounding the prosthetic valve 100 during delivery, such as an outer delivery shaft 224, may be subjected to similar risks. Further, in some cases, apices 114 of the frame 106 may come into contact with the native anatomy of the patient, either along the delivery route to or at the target implantation site.

To address the above-described issues with such W-shaped, U-shaped or more pointed apices that may abrade, puncture and tear an inflatable balloon when the prosthetic valve is crimped thereover, and/or reduce the risk of causing degradation to a delivery sheath (or other surrounding shaft) during delivery of the device to a target implantation site, the prosthetic valve can further comprise protective covers disposed over the end and the side portions of at least some of the apices.

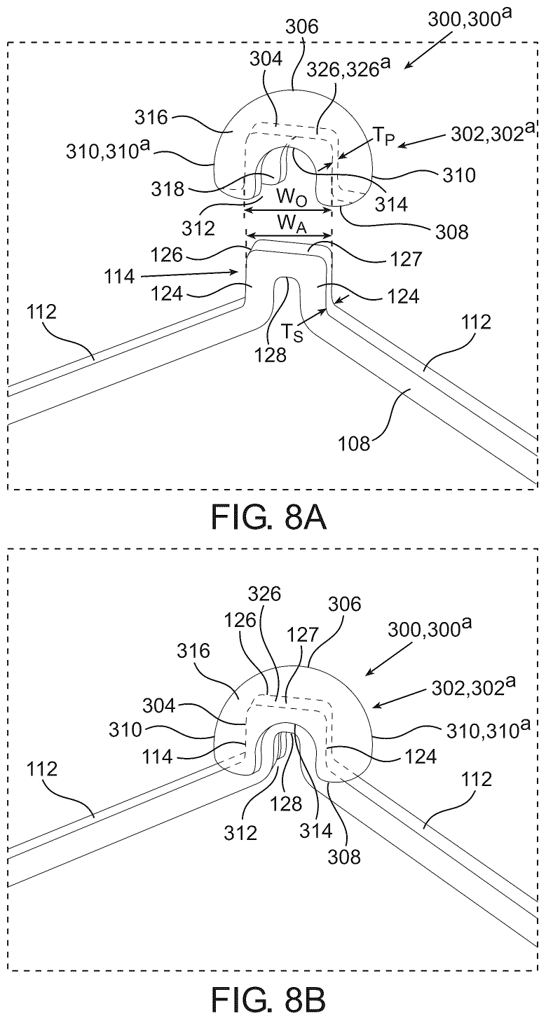

FIGS. 8A-15B show various examples of apex coverings 300 configured to cover apices 114. An apex covering 300 comprises a main body 302 defining an inner pocket 304. The main body 302 extends between a first end 306 and a second end 308 opposite to the first end 306, and includes two main body sidewalls 310 extending between the first end 306 and the second end 308. The first end 306 is a closed end of the apex covering 300, while the second end 308 comprises an opening 312 from which the inner pocket 304 extends toward the first end 306.

The inner pocket 304 can be sized and shaped to fit around a corresponding apex 114. FIG. 8A shows an apex covering 300 positioned next to an apex 114, prior to covering the apex 114, and FIG. 8B shows the apex covering 8B disposed over and coupled to the apex 114. While a U-shaped apex, similar to apex 114^{b} of FIG. 2, is shown throughout FIGS. 8A-14, it is to be understood that this is shown by way of illustration and not limitation and that apex coverings 300 disclosed herein can be used to cover any other type of apex 114.

In some examples, a plurality of apex coverings 300 can be coupled to a plurality of apices 114, such that each discrete apex covering 300 is coupled to and covers at least a portion of a corresponding apex 114. Apex covering 300 can be configured to completely cover at least the end portion 126 of the corresponding apex 114. In some examples, apex coverings 300 are coupled to inflow apices 118. In some examples, apex coverings 300 are coupled to outflow apices 116. In some examples, each inflow apex 118 and each outflow apex 116 includes a discrete apex covering 300 attached thereto. When an apex covering 300 is coupled to an inflow apex 118, the first end 306 can be a distal end of the main body 302, and the second end 308 can be its proximal end. When an apex covering 300 is coupled to an outflow apex 116, the first end 306 can be a proximal end of the main body 302, and the second end 308 can be its distal end. The inner pocket 304 can define a pocket closed end 326, opposite to the opening 312 and closer to the first end 306 of the main body 302.

FIG. 8A shows an apex covering 300 prior to attachment thereof to the corresponding apex 114, while FIG. 8B shows the apex covering 300 disposed or assembled on the apex 114. It is to be understood that an apex 114 to which an apex covering 300 can be attached as shown throughout FIGS. 8A-15B can be an inflow apex 118 or an outflow apex 116. Apex covering 300 defines a covering internal surface 318, facing and optionally contacting the apex 114 when apex covering 300 is assembled thereon, and a covering external surface 316 opposite to the internal surface 318, facing away from the apex 114.

The covering external surface 316 can be a smooth low-friction surface, to avoid damaging other components, such as a balloon 250 or sheaths covering prosthetic valve 100, by otherwise exposed apices 114. In some examples, the apex covering 300 is made of a lubricious and/or low-friction material. In some examples, the main body 302 is coated by a lubricious and/or low-friction material. Appropriate lubricious and/or low-friction material can include, but are not limited to, Teflon, parylene, PTFE, polyethylene, polyvinylidene fluoride, and combinations thereof. In some examples, the covering external surface 316 has a coefficient of friction of 0.1 or less.

Surface roughness is a component of surface texture. It is quantified by the deviations in the direction of the normal vector of a real surface from its ideal form. If these deviations are large, the surface is considered rough, and if they are small, the surface is considered smooth. Therefore, the term “smooth”, as used herein refers to a surface having minor deviations in the direction of the normal vector of a real surface from its ideal form. Smooth surfaces are substantially unitary/continuous surfaces, free from irregular voids. The term “smooth” is not intended to be limited to the narrow meaning of a substantially planar surface devoid of surface irregularities.

Surface roughness is typically calculated by a method termed “Ra” or roughness average, which represents the arithmetic average of a set of individual measurements of surfaces peaks and valleys (e.g., normal vectors), relative to a mean line (e.g., a real surface), wherein low Ra values represents smooth surfaces.

In some examples, the covering external surface 316 or a portion thereof (e.g., the portion of the covering external surface 316 at the first end 306) is characterized by having a Ra value of about 0.2 μm or less, which corresponds to a roughness grade number of N4 (for example, as indicated by ISO 1302:1992). In further examples, the Ra value of the covering external surface 316 or a portion thereof is below about 0.2 μm. In further examples, covering external surface 316 is characterized by having a smooth surface having a Ra value of about 0.2 μm or less.

The covering external surface 316 at the region of the first end 306 is curved, and in some examples, the main body 302 can be substantially spherical or semi-spherical in shape. The curvature of the first end 306 can provide an atraumatic surface, in contrast to the sharper nature of the apex 114 when exposed.

As shown in FIG. 8A, attachment of the apex covering 300 to the apex 8B can be performed, in some examples, by positioning the covering 300 such that opening 312 is facing the end portion 126 of the apex 114, and axially pushing the covering 300 toward and over the apex 114 such that the apex 114 is passed through the opening 312 and received within inner pocket 304, as shown in FIG. 304. This can be achieved either by pushing the apex covering 300 toward apex 114, pushing the frame with apex 114 into the inner pocket 304 of the covering 300, or both.

The opening 312 at the second end 308 of the covering 300 defines an opening width W_{O}, measured as the distance between the circumferentially opposing ends of the opening 312. The inner pocket 304 defines a pocket radial dimension T_{P}, measured as the radial distance between the radially opposing ends of the opening 312 and/or inner pocket 304, at the surfaces facing the frames inner and outer surfaces 108, 110 when the apex covering 300 covers the apex 114.

The opening width W_{O} can correspond to the apex width W_{A}, such as being with a range of up to 15% or less of each other. Pocket radial dimension T_{P} can correspond to the apex thickness T_{A}, such as being with a range of up to 15% or less of each other. In some examples, the opening width W_{O} is substantially equal to the apex width W_{A}, to allow insertion of apex 114 into pocket 304 through opening 312. Similarly, in some examples, the pocket radial dimension T_{P} is substantially equal to the apex thickness T_{A}, to allow insertion of apex 114 into pocket 304 through opening 312.

In some examples, the opening width W_{O} is slightly greater than the apex width W_{A}, such as being up to 15% greater than apex width W_{A}, up to 10% greater than apex width W_{A}, or up to 5% greater than apex width W_{A}. Providing an opening 312 which is slightly wider than the apex 114 can facilitate easier insertion of the apex 114 into the pocket 304. Attachment of apex covering 300 to apex 114 can be achieved by various procedures, such as gluing the covering internal surface 318 to the apex 114, ultrasonic welding, and the like. In some examples, the apex end surface 127, and optionally other surfaces of apex 114, can be textured, such as by being implemented as coarse or rough surfaces, so as to improve adherence to the internal surface 318 of the pocket 304 when an adhesive is applied there-between.

In some examples, the opening width W_{O} is slightly smaller than the apex width W_{A}, such as being up to 15% less than apex width W_{A}, up to 10% less than apex width W_{A}, or up to 5% less than apex width W_{A}. In such examples, the apex covering 300 can comprise a flexible and/or resiliently expandable material, at least around the inner pocket 304, which can allow the opening 312, as well as the remainder of inner pocket 304, to expand to the width W_{A} of the apex 114 when forcibly pushed there-against. Providing an opening 312 which is slightly narrower than the apex 114 can improve retention between both components, as the apex covering 300 snugly covers and is press-fit against the apex 114 residing inside pocket 304.

In some examples, the pocket radial dimension T_{P} is slightly greater than the apex thickness T_{A}, such as being up to 15% greater than apex thickness T_{A}, up to 10% greater than apex thickness T_{A}, or up to 5% greater than apex thickness T_{A}. Providing an opening 312 which is slightly greater in the radial dimension than the thickness of apex 114 can facilitate easier insertion of the apex 114 into the pocket 304.

In some examples, the pocket radial dimension T_{P} is slightly smaller than the apex thickness T_{A}, such as being up to 15% less than apex thickness T_{A}, up to 10% less than apex thickness T_{A}, or up to 5% less than apex thickness T_{A}. In such examples, the apex covering 300 can comprise a flexible and/or resiliently expandable material as mentioned above, at least around the inner pocket 304, so as to allow the opening 312, as well as the remainder of inner pocket 304, to expand to the thickness T_{A} of the apex 114 when forcibly pushed there-against. Providing an opening 312 which is slightly smaller in the radial dimension than the apex 114 can similarly improve retention between both components.

In some examples, the inner pocket 304 and the apex 114 are similarly shaped, such that when the apex covering 300 is disposed over apex 114, the pocket closed end 326 can be in contact with the end portion 126 of the apex 114, and the surfaced along the sides of the pocket 304, extending toward opening 312, can be in contact with the side portions 124 of apex 114. In some examples, main body 302 further comprises a covering depression, which can be aligned with the inner depression 128 of the apex 114.

FIGS. 8A-8B show an apex covering 300^{a}. Apex covering 300^{a} is an exemplary implementation of apex covering 300, and thus includes all of the features described for apex covering 300^{a} throughout the current disclosure, except that the covering external surface 316 of main body 302^{a} is shown to be relatively smooth, without including any grooves or other geometrical features configured to assist in attachment of the covering 300 to the apex 114. In such cases, attachment of main body 302^{a} to apex 114 can be optionally achieved by bonding the covering internal surface 318 along inner pocket 304 to the apex 114, such as by gluing, ultrasonic welding, and the like. As mentioned above, in some examples, the apex end surface 127, and optionally other surfaces of apex 114, can be textured, such as by being implemented as coarse or rough surfaces. Similarly, in some examples, the covering internal surface 318, such as along at least a portion of the inner pocket 304, can be textured, such as by being implemented as a coarse or rough surface, so as to improve adherence to the internal surface 318 of the pocket 304 when an adhesive is applied there-between.

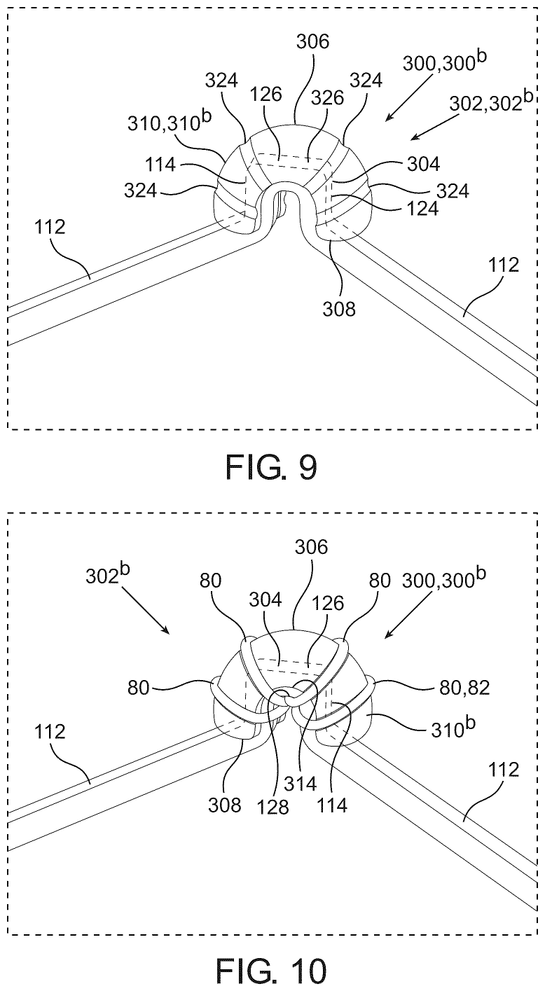

FIG. 9 shows an apex covering 300^{b}. Apex covering 300^{b} is an exemplary implementation of apex covering 300, and thus includes all of the features described for apex covering 300^{b} throughout the current disclosure, except that the main body 302^{b} further includes at least one groove 324 formed at the covering external surface 316. While four grooves 324 are illustrated in FIG. 9, it is to be understood that any other number of grooves is contemplated. While grooves 324 are shown to be formed over the external surface 316 of main body sidewalls 310^{b}, it is to be understood that one or more grooves can be similarly formed along the first end 306. The grooves 324 over main body 302^{b} can be sized and shaped to accommodate sutures or other adequate retention members therein.

FIG. 10 shows the apex covering 300^{b} of FIG. 9, coupled to the apex 114 by one or more sutures 80 looped therearound and received in the groove(s) 324 of main body 302^{b}. Suture 80 can form one or more loops 82, each loop disposed over a separate corresponding groove 324. In some examples, when apex covering 300^{b} includes a plurality of grooves 324, a plurality of sutures 80 can be utilized to form separate loops 82 around each of the corresponding grooves 324. For example, each loop 82 can be formed by surrounding the groove 324 and the corresponding portion of the frame 106 (such as apex 114), and tying the ends of the suture to each other to tightly close the suture 80 in the form of a loop 82, such that a series of independent loops 82 can be formed to couple covering 300^{b} to the apex 114.

In some examples, a single suture 80 can be utilized to form a plurality of loops 82 extending over a corresponding plurality of grooves 324. For example, a single suture 80 can be looped around one of the grooves 324, after which the end of the loops is continuously extended to form a subsequent loop 82 around the neighboring groove 324, and so on. In some examples, when the apex covering 300 includes a covering depression 314 and the apex 114 includes an inner depression 128, the suture 80 can be looped through the covering depression 314 and inner depression 128 of the apex 114, as illustrated in FIG. 10.

It is to be understood that apex coverings 300 disclosed herein can be, in some examples, sutured to apices 114 even if the main bodies 302 are provided without grooves 324. However, the addition of grooves 324 to the main body 302, over which the suture 80 can be looped, can advantageously prevent the suture 80 from slipping away, especially when the covering external surface 316 around main body 302 is provided as a relatively smooth surface. Moreover, it is to be understood that several mechanisms of attachment can be combined and do not necessarily need to be chosen in isolation. For example, the main body 302 of an apex covering 300 can be both sutured to the apex 114 by a suture 80, and glued to the apex 114 by applying an adhesive between the covering internal surface 316 and the apex end surface 127.

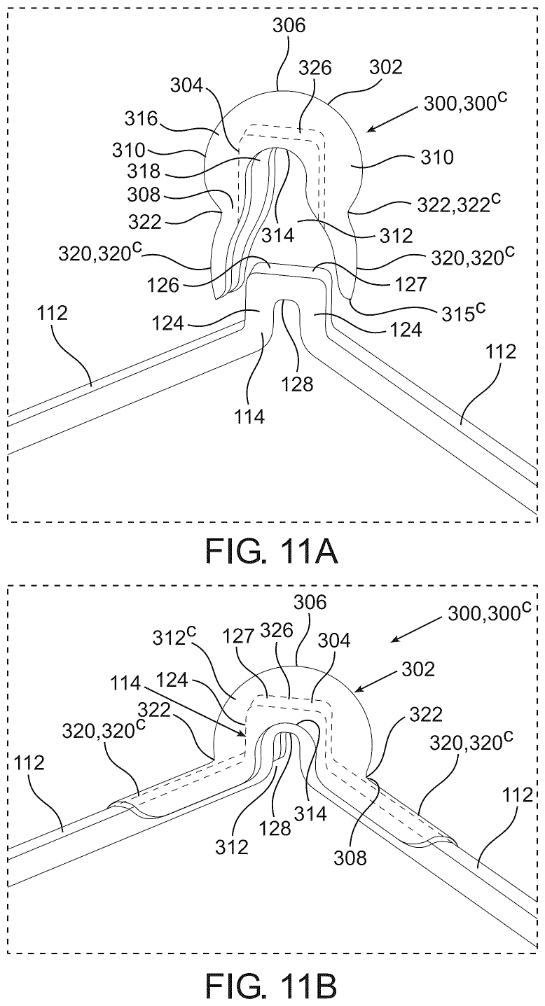

FIGS. 11A and 11B show an apex covering 300^{c}, illustrated prior to attachment to apex 114 and after attachment thereto, respectively. Apex covering 300^{c} is an exemplary implementation of apex covering 300, and thus includes all of the features described for apex covering 300 throughout the current disclosure, except that the main body 302^{c} further includes flexible arms 320 extending from the second end 308 of main body 302. Each arm 320 continuously extends from a corresponding main body sidewall 310, with a joint 322 disposed between the main body sidewall 310 and the arm 320. The joint 322 allows the arms 320 to bend relative to the main body 302, such as relative to the main body sidewall 310 it extends from.

The arms 320 are configured to contact and rest over portions of the struts 112 extending from the apex 114, and more particularly, extending from the side portion 124 of the junction 114, such that when the apex covering 300 is assembled on the apex 114, the joints 322 are aligned with the bent frame transitions between side portions 124 and angled struts 112. The joints 322 can be implemented as compliant hinges or joints formed integrally with the apex covering 300. In some examples, the joints 322 can be implemented as living hinges, in which case the cross-sectional area of the joint 322 can be smaller than that of the arm 320 extending therefrom. The covering internal surface 318 at the region of the arms 320 is the surface facing and contacting struts 112, while the covering external surface 316 at the region of the arms 320 is facing away from the struts 112, such as toward the inflow end 102 when the apex covering 300 is coupled to an inflow apex 118, or toward the outflow end 101 when the apex covering 300 is coupled to an outflow apex 116. In some examples, the covering external surface 316 of arms 320^{c} is relatively smooth, without including any grooves or other geometrical features configured to assist in attachment of the covering 300^{a} to the apex 114.

The arms 320 can provide additional surface area in contact with the frame 106, so as to improve attachment of the apex covering 300 to the apex 114. In some examples, the arms 320 can be relatively flat, configured to contact an axial surface of the struts 112, defines as a surface extending between the frame's inner and outer surfaces 108, 110. In some examples, the arms can be curved, as shown in FIGS. 11A-11B, or can be U-shaped, so as to further contact and cover portions of the inner surface 108 and outer surface 110 of struts 112. coupling of apex covering 300 to apex 114 can be achieved by attaching the arms 320 to the struts 112, either instead of, or in addition to, attaching the main body 302 to the apex 114 according to any example described above. Attachment of the arms 320 to struts 112 can be achieved by bonding the covering internal surface 318 along arms 320 to the struts 112, such as by gluing, ultrasonic welding, and the like. In some examples, the surfaces of the struts 112 configured to contact arms 320 can be textured, such as by being implemented as coarse or rough surfaces. Similarly, in some examples, the covering internal surface 318 along arms 320 can be textured, such as by being implemented as a coarse or rough surface, so as to improve adherence to the internal surface 318 of the arms 320 when an adhesive is applied there-between.

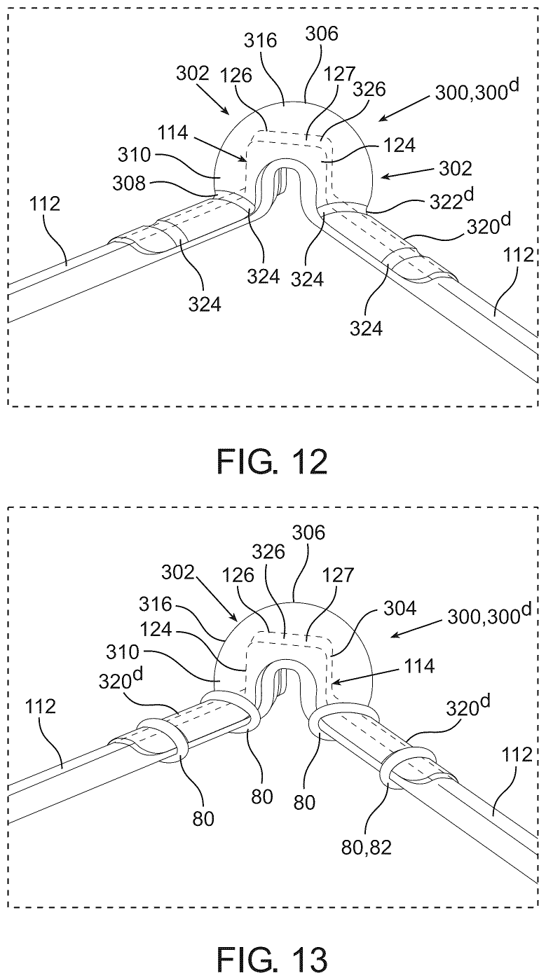

FIG. 12 shows an apex covering 300^{d}. Apex covering 300^{d} is an exemplary implementation of apex covering 300, and thus includes all of the features described for apex covering 300^{d} throughout the current disclosure, and in particular, can include arms 320^{d} in a similar manner to that described above for apex covering 300^{c}, except that apex covering 300^{d} further includes at least one groove 324 formed at the covering external surface 316 at the region of arms 320^{d} and/or joints 322^{d}. While four grooves 324 are illustrated in FIG. 12, one on each arms 320^{d} and one at each joint 3224, it is to be understood that any other number of grooves is contemplated. For example, each arm 320^{d} can include more than one groove 324. Similarly, grooves 324 can be formed at joints 322^{d} while the arms don't include any grooves, or grooves can be formed over arms 320^{d} and not at the joints. The grooves 324 of arms 320^{d} and/or joints 322^{d} can be sized and shaped to accommodate sutures or other adequate retention members therein. Moreover, grooves 324 formed at joints 322^{d} result in thinning of the joints 322^{d} relative to the portions of the arms 320^{d} extending therefrom, in a manner that both allows placement of suture 80 over the grooves 324 of such joints 3224, as well as allowing functionality of such joints 322^{d} as living hinges.

FIG. 13 shows an exemplary attachment of the apex covering 300^{d} of FIG. 12 to the apex 114 by one or more sutures 80 looped around struts 112 and received in the groove 324 of arms 320^{d} and/or joints 3224. Suture 80 can form one or more loops 82, each loop disposed over a separate corresponding groove 324. In some examples, when apex covering 300^{c} includes a plurality of grooves 324, a plurality of sutures 80 can be utilized to form separate loops 82 around each of the corresponding grooves 324. For example, each loop 82 can be formed by surrounding the groove 324 and the corresponding portion of the frame 106 (such as strut 112), and tying the ends of the suture to each other to tightly close the suture 80 in the form of a loop 82, such that a series of independent loops 82 can be formed to couple covering 300^{d} to the apex 114.

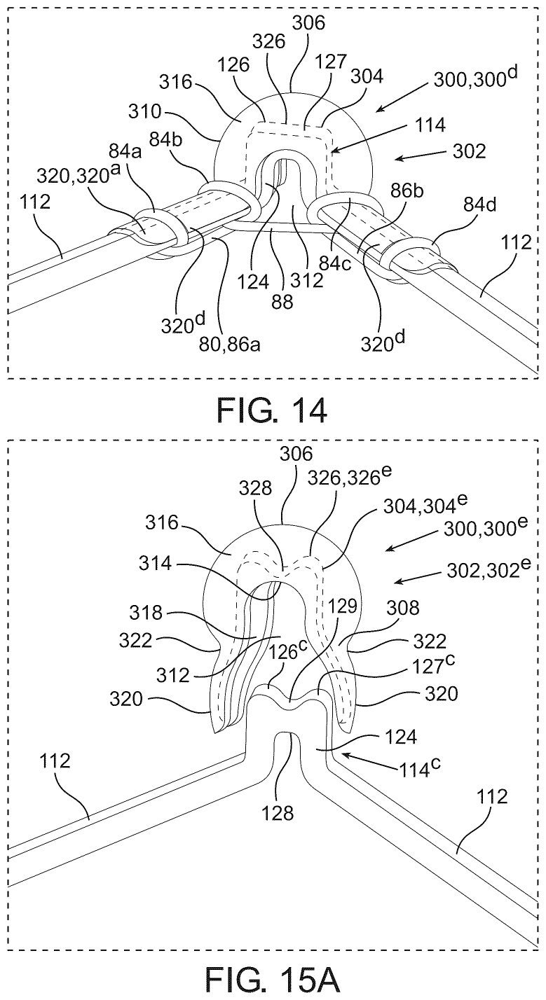

FIG. 14 shows an exemplary attachment of the apex covering 300^{d} of FIG. 12 to the apex 114 by a single suture 80 utilized to form a plurality of loops 84 extending over a corresponding plurality of grooves 324. For example, and as shown in FIG. 14, a single suture 80 can be looped around a strut 112 and one of the grooves 324 of one arm 320^{d} to form a first loop 84a, from which the suture extends along a first transitioning portion 86a, parallel to the corresponding strut 112, toward the subsequent groove 324 formed at joint 3224, around which the suture 80 is looped to form a second loop 84b. The suture 80 can be then passed from second loop 84b toward the groove 324 at the joint 322^{d} opposite to the second loop 84b, forming a crossing portion 88 of the suture 80 extending across the inner depression 128 of apex 114, from one side portion 124 to the opposite side portion 124 of the junction 114, at which point it is looped around the corresponding groove 324 of the target joint 322^{d} so as to form a third loop 84c. The suture then extends from the third loop 84c, along a second transitioning portion 86b parallel to the corresponding strut 112, toward the subsequent groove 324 formed at the corresponding arm 320^{d}, around which the suture 80 is looped to form a fourth loop 84b. Depending on the number and position of grooves 324, the suturing configuration, including the number of loop 84 and transition portions 86, can vary.

It is to be understood that apex coverings 300 with arms 320 disclosed herein can be, in some examples, sutured to apices 114 even if the arms 320 and/or joints 322 are provided without grooves 324. However, the addition of grooves 324 to the arms 320 and/or joints 322, over which the suture 80 can be looped, can advantageously prevent the suture 80 from slipping away, especially when the covering external surface 316 at the region of arms 320 is provided as a relatively smooth surface. Moreover, it is to be understood that several mechanisms of attachment can be combined and do not necessarily need to be chosen in isolation. For example, the arms 320 can be both sutured to the struts 112 by a suture 80, and glued to the struts 112 by applying an adhesive between the covering internal surface 316 of the arms 320 and the corresponding surfaces of the struts 112 facing the arms 320.

It is to be understood that a relatively smooth main body 302 is shown in FIGS. 11A-14 by way of illustration and not limitation, and that any of the apex coverings 300^{c} or 300^{d} can include any exemplary main body 302 described herein, including a smooth main body 302^{a} or a grooved main body 302^{b}. Moreover, it is to be understood that attachment of arms 320 to struts 112 according to any example described herein above, can be performed in combination with a main body 302 attached to the apex 114 according to any example describe above, such as by gluing, suturing, and the like.

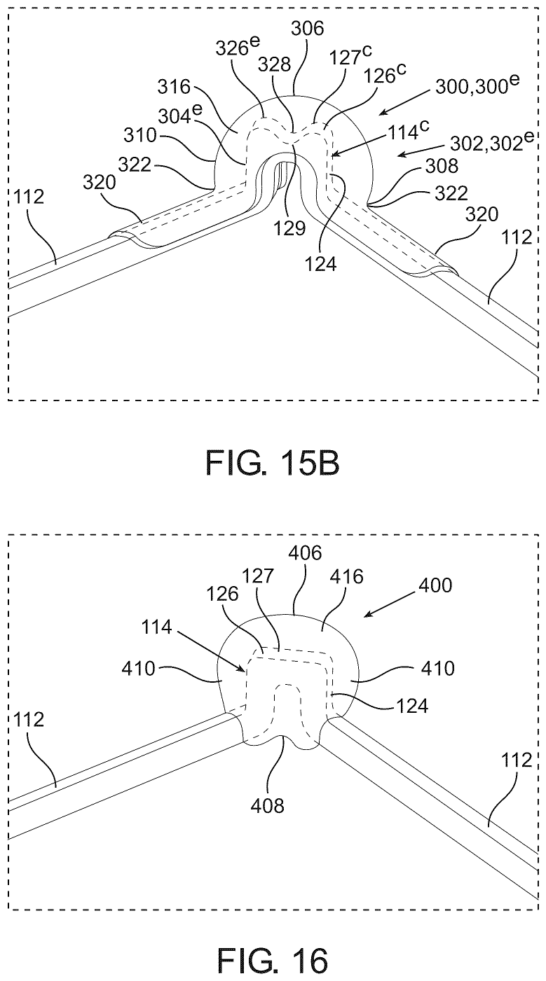

As mentioned above, the inner pocket 304 can correspond in shape to the shape of the apex 114. FIGS. 15A and 15B show an apex covering 300^{e}, illustrated prior to attachment to apex 114^{c} and after attachment thereto, respectively. Apex covering 300^{e} is an exemplary implementation of apex covering 300, and thus includes all of the features described for apex covering 300 throughout the current disclosure, except that the main body the closed end 326^{e} of inner pocket 304^{d} is formed in a shape which is similar to the shape of end portion 126^{c} of apex 114^{c}. For example, the pocket closed end 326^{e} can include a bump 328 configured to contact and be received in the notch 129 of apex 114^{c}. It is to be understood that the shape of inner pocket 304 does not have to match that of the apex 114, for example a U-shaped inner pocket 304 as illustrated in FIGS. 8A-14 can be used to cover an M-shaped or W-shaped apex 114^{c}. However, matching the shapes of the inner pocket 304 and apex 114 can advantageously increase contact area, which in turn can improve attachment between the apex covering 300 and apex 114.

It is to be understood that a smooth main body 302 and smooth arms 320 extending therefrom are shown in Figs. by way of illustration and not limitation, and that apex covering 300^{e} can include any exemplary main body 302 described herein, including a smooth main body 302^{a} or a grooved main body 302^{b}, and can be provided without or with arms 320, which can be in the form of smooth arms 320^{c} or grooved arms 320^{d} and/or joints 322^{d}.

FIG. 16 shows an exemplary apex covering 400 configured to cover apex 114. Apex covering 400 extends between a closed first end 406 closer to the apex end surface 127, and a closed second end 408 opposite to the first end 406, wherein the second end 408 is closer to the struts 112 extending from the corresponding apex 114 than the first end 406. Covering sidewalls 410 can be defined as the portions of the apex covering 400 extending between the first end 406 and the second end 408, along the side portions 124 of apex 114. Apex covering 400 defines a covering external surface 416, facing away from the apex 114 contained inside of apex covering 400. Unlike apex coverings 300 disclosed herein, apex covering 400 does not include an opening 312 at its second end 308 through which apex 114 can be inserted. Thus, both ends 406, 408 of apex covering 400 can be closed ends, such that end portion 126 is enclosed in its entirety within covering 400, optionally with the struts 112 extending through side openings of the covering 400 formed between the covering sidewalls 310 and second end 308. In some examples, apex covering 400 can be formed over apex 114 by coating, such as by dip coating and/or overmolding.

In some examples, the covering external surface 416 or a portion thereof (e.g., the portion of the covering external surface 416 at the first end 306) is characterized by having a Ra value of about 0.2 μm or less, which corresponds to a roughness grade number of N4 (for example, as indicated by ISO 1302:1992). In further examples, the Ra value of the covering external surface 416 or a portion thereof is below about 0.2 μm. In further examples, covering external surface 416 is characterized by having a smooth surface having a Ra value of about 0.2 μm or less.

The covering external surface 416 at the region of the first end 406 is curved, and in some examples, the apex covering 400 can be substantially spherical or semi-spherical in shape. The curvature of the first end 406 can provide an atraumatic surface, in contrast to the sharper nature of the apex 114 when exposed.

Apex coverings disclosed herein, such as any exemplary apex covering 300 or apex covering 400, are configured to prevent damage that may be inflicted by otherwise exposed apices 114 to balloon material during crimping of prosthetic valve 100, as well damage that may occur during axial advancement of prosthetic valve 100 toward balloon 250, such as by push shaft 220, when the valve 100 is crimped at a location proximal to balloon 250. Such coverings 300, 400 can further serve as atraumatic end portions that protect the inner surfaces of other shafts of tubes of the delivery apparatus, surrounding the compressed valve, as axial movement of the valve 100 relative to any surrounding shaft or tube during or after delivery toward the site of implantation, can damage such shaft or tubes due to frictional forces acting on the internal surface of such a shaft by the relatively sharp exposed apices 114 (such as inflow apices 118). However, after implantation within the native annulus, apex coverings 300, 400 no longer serve any role and may actually create flow interferences in their vicinity due to their bulging shape around apices 114. In some examples, apex covering 300 and/or 400 disclosed herein are made of a biodegradable material, configured to degrade or dissolve over time post valve implantation. Biodegradable polymers from which an apex covering 300 and/or 400 is formed can include, but are not limited to: Polyglycolide (PGA), PGA/Polylactide (PLA), PDS-Polydioxanone (PDS), Poly-caprolactone (PCL), Poly(dioxanone), PGA/Tri-Methylene Carbonate (TMC), or combinations thereof.

For cases in which apex coverings 300 made of biodegradable materials, are coupled to the frame 106 by sutures 80, it may be desirable to form the sutures 80 from biodegradable or bioresorbable materials as well, to avoid the sutures 80 loosely hanging around the frame 106 after bio-absorption or bio-degradation of the apex coverings 300. Thus, in some examples, the sutures 80 used for attaching apex coverings 300 to the frame 106, and in particular, to the corresponding apices 114, are made of a biodegradable material configured to degrade or dissolve over time post valve implantation. Biodegradable polymers from which sutures 80 are formed can include, but are not limited to: polylactic acid, polyglycolic acid, polydioxanone, polyethylene glycol, polycapralactone, or combinations thereof.

### SOME EXAMPLES OF THE DISCLOSED IMPLEMENTATIONS

Some examples of above-described implementations are enumerated below. It should be noted that one feature of an example in isolation or more than one feature of the example taken in combination and, optionally, in combination with one or more features of one or more examples below are examples also falling within the disclosure of this application.

Example 1. A prosthetic valve comprising:
a frame configured to transition between a crimped configuration and an expanded configuration, the frame comprising a plurality of intersecting struts and a plurality of apices;
a plurality of apex coverings, each of the apex coverings attached to and covering a corresponding one of the plurality of apices, wherein each apex covering comprises:
   a main body extending between a first end and a second end;
   an opening formed at the second end;
   an inner pocket extending from the opening and sized to accommodate the corresponding apex therein; and
   a covering external surface facing away from the apex covered by apex covering;
wherein the opening is sized to allow insertion of the corresponding apex into the inner pocket.

Example 2. The prosthetic valve of any example herein, particularly example 1, wherein the covering external surface at the region of the first end is curved.

Example 3. The prosthetic valve of any example herein, particularly example 2, wherein the main body is spherical or semi-spherical in shape.

Example 4. The prosthetic valve of any example herein, particularly examples 1 to 3, wherein the plurality of apices comprises a plurality of inflow apices at an inflow end of the prosthetic valve, and wherein the plurality of inflow apices are covered by at least some of the plurality of apex coverings.

Example 5. The prosthetic valve of any example herein, particularly examples 1 to 4, wherein the plurality of apices comprises a plurality of outflow apices at an outflow end of the prosthetic valve, and wherein the plurality of outflow apices are covered by at least some of the plurality of apex coverings.

Example 6. The prosthetic valve of any example herein, particularly examples 1 to 5, wherein each of the plurality of apices covered by the corresponding plurality of apex coverings comprises: two side portions extending from struts to which the apex is attached, and an end portion extending between the two side portions, the end portion defining an apex end surface.

Example 7. The prosthetic valve of any example herein, particularly example 6, wherein the inner pocket comprises a pocket closed end opposite to the opening, the pocket closed end facing the apex end surface.

Example 8. The prosthetic valve of any example herein, particularly example 7, wherein the pocket closed end is in contact with the apex end surface.

Example 9. The prosthetic valve of any example herein, particularly examples 6 to 8, wherein the apex is U-shaped.

Example 10. The prosthetic valve of any example herein, particularly examples 6 to 9, wherein the apex end surface is curved.

Example 11. The prosthetic valve of any example herein, particularly examples 6 to 9, wherein the apex end surface is flat.

Example 12. The prosthetic valve of any example herein, particularly examples 6 to 8, wherein the apex is W-shaped.

Example 13. The prosthetic valve of any example herein, particularly examples 6 to 8, wherein the apex end surface comprises a notch arranged between the side portions.

Example 14. The prosthetic valve of any example herein, particularly example 13, wherein the wherein the pocket closed end comprises a bump configured to contact and be received in the notch.

Example 15. The prosthetic valve of any example herein, particularly examples 1 to 14, wherein the shape of the inner pocket matches the shape of the apex it is attached to.

Example 16. The prosthetic valve of any example herein, particularly examples 1 to 15, wherein the apex defines an apex width in the circumferential direction, wherein the opening defines an opening width in the circumferential direction, and wherein the opening width and the apex width are within a range of 15% of each other.

Example 17. The prosthetic valve of any example herein, particularly examples 1 to 16, wherein the apex defines an apex thickness between an inner surface and an outer surface of the frame, wherein the inner pocket defines a pocket radial dimension, and wherein the pocket radial dimension and the apex thickness are within a range of 15% of each other.

Example 18. The prosthetic valve of any example herein, particularly examples 6 to 8, wherein the main body further comprises at least one groove formed along the covering external surface.

Example 19. The prosthetic valve of any example herein, particularly example 18, wherein the at least one groove of the main body comprises a plurality of grooves.

Example 20. The prosthetic valve of any example herein, particularly examples 18 or 19, wherein the apex covering is attached to the corresponding apex by at least one suture that forms at least one loop extending along the at least one groove of the main body.

Example 21. The prosthetic valve of any example herein, particularly examples 1 to 20, the apex covering further comprises two arms extending from the second end of the main body

Example 22. The prosthetic valve of any example herein, particularly example 21, wherein the arms are attached to struts extending from the apex covered by the apex covering.

Example 23. The prosthetic valve of any example herein, particularly examples 21 or 22, wherein each arm comprises at least one groove formed along the covering external surface.

Example 24. The prosthetic valve of any example herein, particularly examples 23, wherein the at least one groove of each of the arms comprises a plurality of grooves.

Example 25. The prosthetic valve of any example herein, particularly examples 23 or 24, wherein each arm is attached to the corresponding strut by at least one suture that forms at least one loop extending along the at least one groove of the arm.

Example 26. The prosthetic valve of any example herein, particularly examples 21 to 25, wherein each arm is attached to the main body by a joint configured to allow the arm to bend with respect to the main body.

Example 27. The prosthetic valve of any example herein, particularly example 26, wherein the joint is a compliant joint, integrally formed with the arm and the main body.

Example 28. The prosthetic valve of any example herein, particularly examples 26 to 27, wherein each joint comprises a groove formed along the covering external surface.

Example 29. The prosthetic valve of any example herein, particularly example 28, wherein each joint is attached to the frame by at least one suture that forms a loop extending along the groove of the joint.

Example 30. The prosthetic valve of any example herein, particularly examples 1 to 29, wherein the apex covering is made of a biodegradable material.

Example 31. The prosthetic valve of any example herein, particularly examples 1 to 30, wherein the covering external surface has a coefficient of friction of 0.1 or less.

Example 32. The prosthetic valve of any example herein, particularly examples 1 to 31, wherein the covering external surface has a surface roughness Ra value of 0.2 μm or less.

Example 33. The prosthetic valve of any example herein, particularly examples 1 to 14, wherein the apex further comprises an inner depression opposite to the apex end surface.

Example 34. The prosthetic valve of any example herein, particularly example 33, wherein the apex covering further comprises a covering depression which is aligned with the inner depression of the corresponding apex.

Example 35. A method of attaching an apex covering to an apex of a prosthetic valve, comprising:
providing an apex covering that comprises a main body extending between a first end and a second end, the second end comprising an opening from which an inner pocket or the main body extends toward a pocket closed end; and
inserting an apex of a frame of a prosthetic valve, through the opening, into the inner pocket.

Example 36. The method of any example herein, particularly example 35, wherein the apex covering defines a covering external surface which is curved at the region of the first end.

Example 37. The method of any example herein, particularly example 36, wherein the main body is spherical or semi-spherical in shape.

Example 38. The method of any example herein, particularly examples 36 or 37, wherein the covering external surface has a coefficient of friction of 0.1 or less.

Example 39. The method of any example herein, particularly examples 36 to 38, wherein the covering external surface has a surface roughness Ra value of 0.2 μm or less.

Example 40. The method of any example herein, particularly examples 1 to 17, wherein the apex is an inflow apex of the frame

Example 41. The method of any example herein, particularly examples 35 to 39, wherein the apex is an outflow apex of the frame.

Example 42. The method of any example herein, particularly examples 35 to 41, wherein the inserting the apex into the inner pocket comprises pushing the apex covering over the apex.

Example 43. The method of any example herein, particularly examples 35 to 40, wherein the apex comprises two side portions extending from struts to which the apex is attached, and an end portion extending between the two side portions, the end portion defining an apex end surface.

Example 44. The method of any example herein, particularly example 43, wherein the inserting the apex into the inner pocket comprises bringing the apex end surface into contact with the pocket closed end.

Example 45. The method of any example herein, particularly examples 43 or 44, further comprising, prior to the inserting the apex into the inner pocket, applying a layer of glue between the apex end surface and the pocket closed end.

Example 46. The method of any example herein, particularly example 45, wherein the applying a layer of glue comprises applying a layer of glue over the apex end surface.

Example 47. The method of any example herein, particularly examples 45 or 46, wherein the applying a layer of glue comprises applying a layer of glue inside the inner pocket.

Example 48. The method of any example herein, particularly examples 43 to 47, wherein the apex is U-shaped.

Example 49. The method of any example herein, particularly examples 43 to 48, wherein the apex end surface is curved.

Example 50. The method of any example herein, particularly examples 43 to 48, wherein the apex end surface is flat.

Example 51. The method of any example herein, particularly examples 43 to 47, wherein the apex is W-shaped.

Example 52. The method of any example herein, particularly examples 43 to 47, wherein the apex end surface comprises a notch arranged between the side portions.

Example 53. The method of any example herein, particularly example 52, wherein the pocket closed end comprises a bump.

Example 54. The method of any example herein, particularly examples 53, wherein the inserting the apex into the inner pocket comprises positioning the bump inside the notch.

Example 55. The method of any example herein, particularly examples 35 to 54, wherein the shape of the inner pocket matches the shape of the apex it is attached to.

Example 56. The method of any example herein, particularly examples 35 to 55, wherein the main body further comprises at least one groove.

Example 57. The method of any example herein, particularly example 56, further comprising looping at least one suture around the at least one groove of the main body and the frame, so as to attach the apex covering to the apex.

Example 58. The method of any example herein, particularly examples 35 to 57, wherein the apex covering further comprises two arms extending from the second end of the main body.

Example 59. The method of any example herein, particularly example 58, wherein the arms are configured to bend relative to the main body.

Example 60. The method of any example herein, particularly examples 58 or 59, wherein the inserting the apex into the inner pocket comprises placing the arms over struts extending from the apex.

Example 61. The prosthetic valve of any example herein, particularly example 60, wherein each of the arms comprises at least one groove.

Example 62. The method of any example herein, particularly example 61, further comprising looping at least one suture around the at least one groove of the arms and the corresponding strut, so as to attach the arm to the frame.

Example 63. The method of any example herein, particularly examples 58 to 62, wherein the apex covering further comprises a joint disposed between each of the arms and the main body, wherein the joint comprises a groove.

Example 64. The method of any example herein, particularly examples 63, further comprising looping at least one suture around the groove of the joint and the frame.

Example 65. The method of any example herein, particularly examples 43 to 54, wherein the apex further comprises an inner depression opposite to the apex end surface.

Example 66. The method of any example herein, particularly example 65, wherein the apex covering further comprises a covering depression.

Example 67. The method of any example herein, particularly example 66, wherein the inserting the apex into the inner pocket comprises aligning the covering depression with the inner depression of the apex.

Example 68. A prosthetic valve comprising:
a frame configured to transition between a crimped configuration and an expanded configuration, the frame comprising a plurality of apices;
a plurality of apex coverings, each of the apex coverings attached to and covering a corresponding one of the plurality of apices, wherein each apex covering comprises:
   a closed first end;
   a closed second end; and
   a covering external surface facing away from the apex disposed inside the apex covering;
wherein the apex covering is formed from a biodegradable material.

Example 69. The prosthetic valve of any example herein, particularly example 68, wherein the apex covering is formed by dip coating the apex.

Example 70. The prosthetic valve of any example herein, particularly example 68, the apex covering is overmolded around the apex.

Example 71. The prosthetic valve of any example herein, particularly examples 68 to 70, wherein the covering external surface at the region of the first end is curved.

Example 72. The prosthetic valve of any example herein, particularly any one of examples 68 to 71, wherein the main body is spherical or semi-spherical in shape.

Example 73. The prosthetic valve of any example herein, particularly examples 68 to 72, wherein the plurality of apices comprises a plurality of inflow apices at an inflow end of the prosthetic valve, and wherein the plurality of inflow apices are covered by at least some of the plurality of apex coverings.

Example 74. The prosthetic valve of any example herein, particularly examples 68 to 73, wherein the plurality of apices comprises a plurality of outflow apices at an outflow end of the prosthetic valve, and wherein the plurality of outflow apices are covered by at least some of the plurality of apex coverings.

Example 75. The prosthetic valve of any example herein, particularly examples 68 to 74, wherein the covering external surface has a coefficient of friction of 0.1 or less.

Example 76. The prosthetic valve of any example herein, particularly examples 68 to 75, wherein the covering external surface has a surface roughness Ra value of 0.2 μm or less.

It is appreciated that certain features of the disclosure, which are, for clarity, described in the context of separate examples, may also be provided in combination in a single example. Conversely, various features of the disclosure, which are, for brevity, described in the context of a single example, may also be provided separately or in any suitable sub-combination or as suitable in any other described example of the disclosure. No feature described in the context of an example is to be considered an essential feature of that example, unless explicitly specified as such.

In view of the many possible examples to which the principles of the disclosure may be applied, it should be recognized that the illustrated examples are only preferred examples and should not be taken as limiting the scope. Rather, the scope is defined by the following claims. We therefore claim all that comes within the scope and spirit of these claims.

1. A prosthetic valve comprising:
a frame configured to transition between a crimped configuration and an expanded configuration, the frame comprising a plurality of intersecting struts and a plurality of apices;
a plurality of apex coverings, each of the apex coverings attached to and covering a corresponding one of the plurality of apices, wherein each apex covering comprises:
a main body extending between a first end and a second end;
an opening formed at the second end;
an inner pocket extending from the opening and sized to accommodate the corresponding apex therein; and
a covering external surface facing away from the apex covered by apex covering;
wherein the opening is sized to allow insertion of the corresponding apex into the inner pocket.

2. The prosthetic valve of claim 1, wherein the plurality of apices comprises a plurality of inflow apices at an inflow end of the prosthetic valve, and wherein the plurality of inflow apices are covered by at least some of the plurality of apex coverings.

3. The prosthetic valve of claim 1, wherein each of the plurality of apices covered by the corresponding plurality of apex coverings comprises: two side portions extending from struts to which the apex is attached, and an end portion extending between the two side portions, the end portion defining an apex end surface.

4. The prosthetic valve of claim 3, wherein the apex end surface is curved.

5. The prosthetic valve of claim 3, wherein the apex is W-shaped.

6. The prosthetic valve of claim 3, wherein the apex end surface comprises a notch arranged between the side portions.

7. The prosthetic valve of claim 6, wherein the inner pocket comprises a pocket closed end opposite to the opening, and wherein the pocket closed end comprises a bump configured to contact and be received in the notch.

8. The prosthetic valve of claim 1, wherein the main body further comprises at least one groove formed along the covering external surface.

9. The prosthetic valve of claim 8, wherein the apex covering is attached to the corresponding apex by at least one suture that forms at least one loop extending along the at least one groove of the main body.

10. The prosthetic valve of claim 1, wherein the apex covering further comprises two arms extending from the second end of the main body.

11. The prosthetic valve of claim 10, wherein the arms are attached to struts extending from the apex covered by the apex covering.

12. The prosthetic valve of claim 10, wherein each arm comprises at least one groove formed along the covering external surface.

13. The prosthetic valve of claim 10, wherein each arm is attached to the main body by a joint configured to allow the arm to bend with respect to the main body.

14. The prosthetic valve of claim 1, wherein the apex covering is made of a biodegradable material.

15. A method of attaching an apex covering to an apex of a prosthetic valve, comprising:
providing an apex covering that comprises a main body extending between a first end and a second end, the second end comprising an opening from which an inner pocket or the main body extends toward a pocket closed end; and
inserting an apex of a frame of a prosthetic valve, through the opening, into the inner pocket.

16. The method of claim 15, wherein the main body further comprises at least one groove.

17. The method of claim 16, further comprising looping at least one suture around the at least one groove of the main body and the frame, so as to attach the apex covering to the apex.

18. A prosthetic valve comprising:
a frame configured to transition between a crimped configuration and an expanded configuration, the frame comprising a plurality of intersecting struts and a plurality of apices;
a plurality of apex coverings, each of the apex coverings attached to and covering a corresponding one of the plurality of apices, wherein each apex covering comprises:
a closed first end;
a closed second end; and
a covering external surface facing away from the apex disposed inside the apex covering;
wherein the apex covering is formed from a biodegradable material.

19. The prosthetic valve of claim 18, wherein the main body is spherical or semi-spherical in shape.

20. The prosthetic valve of claim 18, wherein the plurality of apices comprises a plurality of inflow apices at an inflow end of the prosthetic valve, and wherein the plurality of inflow apices are covered by at least some of the plurality of apex coverings.
